(19) United States
(12) Patent Application Publication
(10) Pub. No.: US 2026/0224740 A1
(43) Pub. Date: Aug. 6, 2026

(54) PHARMACEUTICAL FORMULATIONS OF GENE DELIVERY VEHICLES

(71) Applicant: UNIQURE BIOPHARMA B.V., Amsterdam (NL)

(72) Inventors: Maria Mihaela APETRI, Amsterdam (NL); Gabriel FAIMAN, Amsterdam (NL); Edina LJUBOVIC-COUTEAU, Amsterdam (NL); Eddy BERTHIER, Amsterdam (NL)

(73) Assignee: UNIQURE BIOPHARMA B.V., Amsterdam (NL)

(21) Appl. No.: 19/148,030

(22) PCT Filed: Jan. 19, 2024
(86) PCT No.: PCT/EP2024/051201

§ 371 Date: Jul. 15, 2025

(30) Foreign Application Priority Data
Jan. 19, 2023 (EP) 23152382.0

(51) Int. Cl. A61K 48/00; A61K 47/02; A61K 47/18; A61K 47/20; A61K 47/22; A61K 47/26; A61K 47/40; A61P 25/28

(52) U.S. Cl. CPC A61K 48/0033; A61K 47/02; A61K 47/183; A61K 47/20; A61K 47/22; A61K 47/26; A61K 47/40; A61P 25/28

(57) ABSTRACT

The invention relates to formulations comprising miRNA that have improved stability for the treatment of diseases including neurodegenerative diseases such as Huntington's disease.

### TECHNICAL FIELD

The invention relates to the field of gene therapy. In addition, the invention relates to the field of interfering RNA and/or microRNA (miRNA). In particular, the invention relates to gene therapy involving such miRNA and more in particular to pharmaceutical formulations with improved stability for the treatment of diseases including neurodegenerative diseases such as Huntington's disease.

### BACKGROUND OF THE INVENTION

### Gene Therapy

The elucidation of DNA as the carrier of genetic information, and therefore also as the source of inherited diseases, has led to envisaged therapies in which mutant, damaged genes could be replaced or at least silenced. Many genes and/or other nucleic acid sequences have now been identified to play a role in (genetic) disease. If the mutant gene(s) could be replaced by a healthy one, or if the genes expressing aberrant (sometimes toxic) products could be silenced, the disease could be treated at the molecular level and potentially be cured. Gene therapy provides a promising concept, in particular for diseases caused by mutations in a single gene.

However, the delivery of the desired nucleic acid to the cells that need to be targeted is not an easy task. Numerous (viral) delivery systems have been investigated, all of them with their advantages and drawbacks. One of the viral delivery vehicles that are used for gene therapy is the Adeno-Associated Virus (AAV).

### AAV

AAV has a single-stranded DNA genome of approximately 4.8 kilobases (kb). AAV belongs to the parvovirus family and is dependent for replication on co-infection with other viruses, in particular adenoviruses. The genome comprises Rep (Replication) and Cap (Capsid) genes. These coding sequences are flanked by inverted terminal repeats (ITRs) that are required for genome replication and packaging. The Rep gene encodes four proteins (Rep78, Rep68, Rep52, and Rep40), replicates the viral genome, and facilitates packaging, while Cap expression gives rise to the viral capsid proteins (VP; VP1 VP2 VP3), which form the outer capsid shell.

For gene therapy, the viral DNA of the AAV is almost completely removed. Recombinant AAV (rAAV) for gene therapy is formed by a protein capsid containing a desired nucleic acid, the transgene, that is to be delivered to target cells. The desired nucleic acid is flanked by the ITRs of AAV. ITR-flanked transgenes encoded by rAAV can form circular concatemers remaining in the nucleus of transduced cells as episomes. As the episome remains largely episomal, the expression of AAV delivered nucleic acid sequences may be diluted over time if and when the target cell replicates. This dilution may not generally apply to post-mitotic cells such as neurons, which are the target cells for many neurodegenerative diseases.

miRNA

RNA interference (RNAi) is a naturally occurring mechanism that involves sequence-specific down-regulation of messenger RNA (mRNA). The down-regulation of mRNA results in a reduction of the amount of protein that is expressed. RNA interference is triggered by double-stranded RNA. One of the strands of the double-stranded RNA is substantially or completely complementary to its target, the mRNA. This strand is termed the guide strand. The mechanism of RNA interference involves the incorporation of the guide strand in the RNA-induced silencing complex (RISC). This complex is a multiple turnover complex that via complementary base pairing of the guide strand can bind to its target mRNA. Once bound to its target mRNA it can either cleave the mRNA or reduce translation efficiency. RNA interference has since its discovery been widely used to knock down specific target genes. The triggers for inducing RNA interference that have been employed involve the use of small interfering RNAs (siRNAs) or short hairpin RNAs (shRNAs). In addition, molecules that can naturally trigger RNAi, the so-called microRNAs (miRNAs), have been used to make artificial (engineered) miRNAs that mimic their naturally occurring counterparts. These strategies have in common that they provide for substantially double-stranded RNA molecules that are designed to target an mRNA of choice. RNAi-based therapeutic approaches that utilize the sequence-specific modality of RNAi are under development.

### Huntington's Disease

The huntingtin gene, also referred to as the HTT or HD (Huntington's disease) gene, encodes for the huntingtin mRNA and protein. The huntingtin gene is a large gene on chromosome 4p.l3 of about 13.5 kb (huntingtin protein is about 350 kDa). Huntington's disease is a genetic neurodegenerative disorder caused by a genetic mutation in the huntingtin gene. The genetic mutation involves a DNA segment of the huntingtin gene known as the CAG trinucleotide repeat. Normally, the CAG segment in the huntingtin gene of humans is repeated multiple times, i.e. about 10-35 times. People that develop

Huntington's disease have an expansion of the number of CAG repeats in at least one allele. An affected person usually inherits the mutated allele from one affected parent. In rare cases, an individual with Huntington's disease does not have a parent with the disorder (sporadic HD). People with 36 to 39 CAG repeats may develop signs and symptoms of HD, while people with 40 or more repeats always develop the disorder, marked by a triad of motor, cognitive and psychiatric symptoms that ultimately leads to death. The increase in the size of the CAG repeat leads to the production of an aberrant HTT mRNA resulting in an RNA toxic gain-of-function, and to a production of mutant huntingtin protein with an elongated polyglutamine (poly Q) stretch. The mutant huntingtin protein is processed in the cell into smaller fragments that are cytotoxic and that accumulate and aggregate in neurons, starting in the striatum and the cerebral cortex in later stages of the disease. This results in the disruption of normal function and the eventual death of neurons. This is the main process that occurs in the brain which underlies the signs and symptoms of Huntington's disease.

According to the European Medicines Agency (EMA), Huntington's disease affects approximately 70,000 people in the U.S. and Europe, making it one of the largest clinical unmet needs in the rare disease field. Despite the known etiology of the disease, there are no therapies currently available to treat the disease, delay its onset, or slow the progression of a patient's decline.

### Formulation and Administration Challenges

Using AAV vectors to deliver micro-RNAs directly to a patient's brain for non-selective knockdown of the huntingtin gene could represent a highly innovative and promising approach for treating Huntington's disease. Initial studies have demonstrated that a single administration of an AAV vector may result in a dose-dependent and sustained reduction of mutant huntingtin protein (mHTT) in the deep structures of the brain-including the striatum and the putamen, where Huntington's disease is known to manifest, and the cortex. Once administered, the AAV vectors are observed to spread to the cerebral cortex and lowered mHTT in the frontal areas of the brain that show neuropathological changes later in the course of the disease, providing evidence of spread from the injection sites to the cerebral cortex.

However, administration directly to a patient's brain presents special challenges for the formulation and stability of a suitable pharmaceutical formulation, such as the need to avoid undesirable formation of aggregated or agglomerated particles in the formulation that may be detrimental to a patient. In particular, such formulations may be stored frozen and then thawed before use. Aggregates or agglomerates could problematically form during or after the thaw of the formulation, and also during the time following thawing but before administration of the formulation. It would therefore be highly desirable to provide an innovative formulation of an AAV vector for treating Huntington's disease, or other diseases, that eliminates or substantially eliminates aggregate or agglomerate formation for extended in-use shelf life.

### SUMMARY OF INVENTION

In a first aspect there is provided an isotonic pharmaceutical formulation comprising: a buffer; an adeno-associated viral vector with a transgene encoding a microRNA; and a cyclodextrin or a derivative thereof.

In some embodiments, the formulation has an osmolarity of from 250 to 330 mOsm/kg and a pH value of from 6.5 to 8.

In some embodiments, the formulation is essentially free of visible particles.

In some embodiments, the adeno-associated viral vector with a transgene encoding a microRNA targets huntingtin mRNA.

In some embodiments, the adeno-associated viral vector comprises an AAV2 serotype, an AAV5 serotype, an AAV9 serotype, a hybrid AAV serotype, or a combination thereof.

In some embodiments, the concentration of the adeno-associated viral vector is up to 5E13 gc/ml.

In some embodiments, the cyclodextrin is an unsubstituted or substituted β-cyclodextrin at a concentration of from about 0.05% w/v to about 2% w/V.

In some embodiments, the formulation further comprises a sugar or sugar alcohol at a concentration of from about 0.05% w/v to about 2% w/v.

In some embodiments, the sugar or sugar alcohol is selected from the group consisting of trehalose, sucrose, maltose, mannitol, and derivatives and combinations thereof.

In some embodiments, the formulation further comprises a pharmaceutically acceptable salt selected from the group consisting of NaCl, KCl, CaCl₂), MgCl₂, and combinations thereof, at a concentration of greater than 75 mM

In some embodiments, the buffer comprises a Tris buffer at a pH of about 7.5 to about 8.0.

In some embodiments, the formulation further comprises an amino acid selected from the group consisting of cysteine, arginine, histidine, glycine, and derivatives and combinations thereof, at a concentration of from about 2 mM to about 3 mM.

In some embodiments, the formulation remains stable at room temperature (15° C.-25° C.) without substantial formation of aggregates or agglomerates for at least 12 hours after thawing.

In a second aspect, there is provided the formulation of the invention for use in treating Huntington's disease.

In some embodiments, the formulation is administered to a patient's brain by intrastriatal injection targeting the putamen and caudate nucleus.

### DETAILED DESCRIPTION OF INVENTION

The present invention seeks to provide a new pharmaceutical formulation that has reduced aggregate or agglomerate formation during processing and is essentially free of visible particles for an extended time at room temperature after thawing of the formulated drug product. The present invention also seeks to provide a pharmaceutical formulation comprising one or more excipients that is substantially isotonic to human cerebrospinal fluid (CSF) for improved administration to a patient's brain. The present invention also seeks to provide a method of treating Huntington's disease by administering the formulation of the invention to a patient's brain.

In one aspect of the invention, the present invention provides an isotonic pharmaceutical formulation comprising a buffer, an adeno-associated viral vector with a transgene encoding a microRNA, and a cyclodextrin or a derivative thereof.

In this way, it has been surprisingly found that the addition of a cyclodextrin or a derivative thereof to the formulation of the invention helps to improve the stability of the drug product. The formulation may remain stable without substantial formation of aggregates or agglomerates for at least about 6 months, preferably at least about 12 months, more preferably at least about 24 months, for example at least 36 months, at a storage temperature of ≤−65° C. The formulation may also remain stable at room temperature (15° C.-25° C.) without substantial formation of aggregates or agglomerates for an extended time, preferably at least about 12 hours, more preferably at least about 24 hours, after thawing. Thus, in certain embodiments, the formulation of the invention remains stable at room temperature (15° C.-25° C.) without substantial formation of aggregates or agglomerates for at least 12 hours after thawing.

In some embodiments, the adeno-associated viral vector with a transgene encoding a microRNA may target huntingtin mRNA, which may enable the formulation to treat Huntington's disease. For example, the adeno-associated viral vector with a transgene encoding a microRNA may target exon 1 of huntingtin mRNA, thereby lowering huntingtin mRNA and protein levels. The targeted huntingtin mRNA may be a mutant huntingtin mRNA or a wild-type huntingtin mRNA. Therefore, in some embodiments, the adeno-associated viral vector with a transgene encoding microRNA targets huntingtin mRNA. In 30 further embodiments, the adeno-associated viral vector with a transgene encoding microRNA targets exon 1 of huntingtin mRNA. In certain specific embodiments, the adeno-associated viral vector with a transgene encoding microRNA targets mutant huntingtin mRNA. In other specific embodiments, the adeno-associated viral vector with a transgene encoding microRNA targets wild-type huntingtin mRNA.

In some embodiments, the adeno-associated viral vector comprises an AAV2 serotype, an AAV5 serotype, an AAV9 serotype, a hybrid AAV serotype, or a combination thereof. Said serotypes have been found particularly preferred for the treatment of Huntington's disease. In some embodiments, the adeno-associated viral vector comprises an AAV5 serotype. In one example, the adeno-associated viral vector comprises an AAV5 (765) variant. In some embodiments, the adeno-associated viral vector comprises an AAV9 serotype

In some embodiments, the adeno-associated viral vector comprises a hybrid AAV serotype. By way of example, the hybrid AAV serotype may be a hybrid AAV2/AAV5; AAV2/AAV9; or AAV5/AAV9 serotype. An example of a method and means to deliver miRNA to target cells has been provided in WO2020/104469 A1, which is incorporated herein by reference.

In some embodiments, the concentration of the adeno-associated viral vector is up to 5E13 gc/ml (genome copies per milliliter), preferably up to 2E13 gc/ml. In one example, the formulation comprises an AAV5 with a transgene encoding a microRNA, for example a microRNA targeting huntingtin mRNA, and the concentration of AAV5 is 2E12 gc/ml. In another example, the formulation comprises an AAV5 with a transgene encoding a microRNA, for example a microRNA targeting huntingtin mRNA, and the concentration of AAV5 is 2E13 gc/ml.

In one example, the formulation comprises an AAV9 with a transgene encoding a microRNA, for example a microRNA targeting huntingtin mRNA, and the concentration of AAV9 is 2E12 gc/ml. In another example, the formulation comprises an AAV9 with a transgene encoding a microRNA, for example a microRNA targeting huntingtin mRNA, and the concentration of AAV9 is 2E13 gc/ml.

The isotonic formulation of the invention comprises a cyclodextrin or a derivative thereof. In some embodiments, the cyclodextrin or derivative thereof is selected from the group consisting of α-cyclodextrins, β-cyclodextrins, γ-cyclodextrins, and combinations thereof. Preferably, the cyclodextrin is an unsubstituted or substituted β-cyclodextrin. Without wishing to be bound by theory, the inventors of the present invention have found that, surprisingly, cyclodextrins may contribute to the improvement in the preservation of the adenoviruses, and/or are particularly advantageous in providing a stable drug product that reduces substantially the formation of aggregates or agglomerates. 2-hydroxypropyl-β-cyclodextrin is particularly preferred.

In some embodiments, the cyclodextrin is present in the formulation in an amount less than about 4% w/v (weight per volume percent), preferably less than about 2% w/v, preferably about 0.05% w/v to about 4% w/v, preferably about 0.05% w/v to about 2% w/v, preferably about 0.1% w/v to about 1% w/v. In some specific embodiments of the isotonic formulation of the invention, the cyclodextrin is an unsubstituted or substituted β-cyclodextrin at a concentration of from about 0.05% w/v to about 2% w/v.

In some preferred embodiments of the formulation of the invention, the cyclodextrin is 2-hydroxypropyl-β-cyclodextrin. In some specific embodiments, the 2-hydroxypropyl-β-cyclodextrin is provided in the formulation in an amount less than about 2% w/v; preferably about 1% w/v or less; preferably about 0.05% w/v to about 1% w/v; for example about 0.1% w/v. Formulations with 2-hydroxypropyl-β-cyclodextrin at the above levels are particularly advantageous for administration directly to a patient's brain.

In some embodiments, the formulation further comprises a sugar or sugar alcohol, preferably a monosaccharide, disaccharide, or sugar alcohol. Preferably, the sugar or sugar alcohol may be selected from the group consisting of trehalose, sucrose, maltose, mannitol, and derivatives and combinations thereof. For example, the sugar or sugar alcohol is mannitol. It has been found that, surprisingly, sugar or sugar alcohols may act as cryoprotectants and may be particularly advantageous in providing a stable pharmaceutical formulation.

In some embodiments, the sugar or sugar alcohol is present in the formulation in an amount of about 0.01% w/v to about 4% w/v, preferably about 0.05% w/v to about 2% w/v, preferably about 0.1% w/v to about 1% w/v. Thus, in some embodiments, the isotonic formulation of the invention comprises a sugar or sugar alcohol at a concentration of from about 0.05% w/v to about 2% w/v. In some specific embodiments, the isotonic formulation comprises a sugar or sugar alcohol selected from the group consisting of trehalose, sucrose, maltose, mannitol, and derivatives and combinations thereof, at a concentration of from about 0.05% w/v to about 2% w/v.

Where the sugar or sugar alcohol is mannitol, it may be present in the formulation in an amount less than about 2% w/v, preferably about 1% w/v or less, preferably about 0.05% w/v to about 1% w/v, preferably about 0.1% w/v, which is particularly advantageous in providing a formulation that is suitable for administration directly to a patient's brain. In some embodiments, the isotonic formulation comprises mannitol at a concentration of about 0.1% w/v.

In some embodiments, the formulation comprises the combination of 2-hydroxypropyl-β-cyclodextrin and mannitol, preferably in the amount of about 0.1 w/v % 2-hydroxypropyl-β-cyclodextrin and about 0.1 w/v % mannitol.

Preferably, the formulation of the invention is substantially isotonic to human cerebrospinal fluid. Tonicity is a measure for the effective osmotic pressure that a liquid formulation can exert, and depends primarily on the number of dissolved particles in solution. Osmotic pressure is an important factor affecting biological cells. Hypertonicity is the presence of a solution that causes cells to shrink. Hypotonicity is the presence of a solution that causes cells to swell. Isotonicity is the presence of a solution that produces no change in cell volume. When a biological cell is in a hypotonic environment, the cell interior accumulates water, water flows across the cell membrane into the cell, causing it to expand. For mammalian cells this can lead to cytolysis, and tonicity is therefore important when fragile cells such as nerve cells or brain cells are to be exposed to a composition. Tonicity agents are therefore added to injectable preparations to prevent osmotic shock at the site of injection upon administration, and thereby reduce local irritation or even damage to the CNS. Typical tonicity agents are thus excipients used for tonicity adjustment, and are known in the art.

Preferably, the formulation is substantially isotonic to human cerebrospinal fluid. In particular, the formulation of the invention may have an tonicity of from 250 to 330 mOsm/kg. In some preferred embodiments, the formulation has a tonicity of from 270 to 310 mOsm/kg. For example, the formulation has tonicity of 290 mOsm/kg.

Tonicity agents include, but are not limited to, dextrose, glycerin, mannitol, and metal salts. Thus, in some embodiments, the pharmaceutically acceptable salt may comprise a periodic group 1 or group 2 metal salt, preferably a periodic group 1 or group 2 metal chloride salt, preferably selected from the group consisting of NaCl, KCl, CaCl₂), MgCl₂, and combinations thereof. NaCl is particularly preferred.

The formulation comprises the pharmaceutically acceptable salt at a concentration greater than about 75 mM, which has been found beneficial for the stability of the drug product. Preferably, the concentration of pharmaceutically acceptable salt may be about 75 mM to about 200 mM, preferably about 75 mM to about 150 mM. Thus, in some embodiments, the isotonic formulation of the invention comprises a pharmaceutically acceptable salt selected from the group consisting of NaCl, KCl, CaCl₂), MgCl₂, and combinations thereof, at a concentration of greater than 75 mM.

In some specific embodiments, the pharmaceutically acceptable salt comprises NaCl, present in the formulation at a concentration of about 75 mM or higher, preferably about 75 mM to about 150 mM. The formulation of the invention may have a pH value compatible with human cerebrospinal fluid. For example, the formulation may have a pH of about 6.5 or higher, preferably about 7 or higher. In some embodiments, the formulation may have a pH value of from 6.5 to 8, preferably from 7 to 8, more preferably from 7.3 to 7.7. In some embodiments of the present invention, the formulation has a pH value of 7.5.

Buffering agents are known in the art, and help maintain the pH of the composition stable within a given range. A buffering agent is often a buffer salt. Thus, the formulation of the invention may comprise a buffer selected from the group consisting of acetate, citrate, phosphate, Tris, and derivatives and combinations thereof. For example, the isotonic buffer may be a Tris buffer at a pH of about 7.5 to about 8.0, a citrate buffer at a pH of about 5.5 to about 6.5, or a phosphate buffer at a pH of about 7.0 to about 7.5. Preferably, the buffer is a Tris buffer. In some embodiments, the isotonic formulation comprises a Tris buffer at a pH of about 7.5 to about 8.0. In some specific embodiments, the buffer is a Tris buffer at a pH of 7.5. In some preferred embodiments, the buffer is a 20 mM Tris buffer at a pH of 7.5.

In some embodiments, the isotonic formulation of the invention has an osmolarity of from 250 to 330 mOsm/kg and a pH value of from 6.5 to 8.

In preferred embodiments of the invention, the formulation is substantially isotonic to human cerebrospinal fluid. For example, the formulation has a tonicity of 270 to 310 mOsm/kg and a pH value of 7.1 to 7.7.

In some embodiments, the formulation further comprises an amino acid, which has been found beneficial for the stability of the drug product. For example, the amino acid may be selected from the group consisting of cysteine, arginine, histidine, glycine, and derivatives and combinations thereof. Preferably, the amino acid may comprise histidine and/or glycine. Where the amino acid is histidine and/or glycine, it may be present at a concentration of about 2 mM to about 3 mM, preferably about 2.5 mM.

Thus, in some embodiments, the isotonic formulation comprises an amino acid selected from the group consisting of cysteine, arginine, histidine, glycine, and derivatives and combinations thereof, at a concentration of from about 2 mM to about 3 mM. In some specific embodiments, the isotonic formulation comprises histidine, or a derivative thereof, at a concentration of about 2.5 mM.

In some embodiments, the formulation does not comprise a surfactant. Surfactants and their characteristics are well known; surfactants generally comprise at least one polar head group and at least one apolar or hydrophobic tail and are preferably charge neutral, i.e., they do not have a net charge at the conditions for their use. For example, the formulation does not comprise Polysorbate 20, Polysorbate 80, or Poloxamer 188. It is believed that such a formulation may be advantageous in providing a safe formulation for administration to a patient's brain; whilst certain surfactant has been found to improve stability in certain compositions, their use may be associated to potential risks for the patient.

The buffering agent, tonicity agent, cryoprotectant, and any other functionally defined components of the composition, such as a surfactant, can be individual substances, but can also be mixtures. For instance, the buffering agent can be a single phosphate salt such as Na2HPO4, but it can also be a mixture of substances such as a mixture of Na2HPO4 and KH2PO4. Similarly the tonicity agent can be a single substance such as NaCl, but it can also be a mixture such as a combination of NaCl and KCl. Throughout this document, when only a single substance is specified for a functional definition, preferably no other substances are present for that same function. The same holds for when a plurality is specified, in which case only the specified substances are comprised for that same function.

Some compounds, such as for example phosphate salts, can act as a buffering agent but can also act as a tonicity agent. As used herein, when a compound is present as one functionally defined component of the composition, then it preferably is not considered as also satisfying any requirements for further functionally defined components. Accordingly, when one or more of acetate salts, citrate salts, HEPES, TRIS, or phosphate salts are present, these are preferably considered as buffering agent only. When one or more of NaCl, KCl, MgCl2, CaCl2, or a mixture thereof are present, these are preferably considered as tonicity agent only. When one or more of trehalose, dextran, sucrose, dextrose, or poly(ethylene glycol) are present, these are preferably considered as cryoprotectant only. When one or more non-ionic surfactants are present, these are preferably considered as surfactants only.
In a particularly preferred embodiment, the formulation comprises, consists, or consists essentially of: an adeno-associated viral vector with a transgene encoding a microRNA targeting huntingtin mRNA;
about 10-30 mM Tris at a pH of about 7-8;
about 100-150 mM NaCl;
about 1-4 mM histidine;
about 0.05-0.2% w/v hydroxypropyl-β-cyclodextrin; and
about 0.05-0.2% w/v mannitol.

For example, the formulation may comprise, consist or consist essentially of:
an adeno-associated viral vector with a transgene encoding a microRNA targeting huntingtin mRNA;
about 20 mM Tris at a pH about 7.5;
about 125 mM NaCl;
about 2.5 mM KCl;
about 2.5 mM MgCl₂ or MgSO4;
about 2.5 mM CaCl₂);
about 2.5 mM histidine;
about 0.1% w/v hydroxypropyl-β-cyclodextrin; and
about 0.1% w/v mannitol.

In this way, magnesium sulfate or magnesium chloride, calcium chloride, and potassium chloride may be added to mimic the composition of human cerebrospinal fluid. Isotonicity to human cerebrospinal fluid may be achieved by the addition of 125 mM sodium chloride.

In another example, the formulation may comprise, consist or consist essentially of:
an adeno-associated viral vector with a transgene encoding a microRNA targeting huntingtin mRNA;
about 20 mM Tris at a pH of about 7.5;
about 135 mM NaCl;
about 2.5 mM histidine;
about 0.1% w/v hydroxypropyl-β-cyclodextrin; and
about 0.1% w/v mannitol.

In this way, the formulation may be adapted to increase the robustness of the manufacturing process at scale. For this reason, magnesium sulfate or magnesium chloride, calcium chloride, and potassium chloride may be excluded, while the sodium chloride concentration may be increased to 135 mM to maintain isotonicity to human cerebrospinal fluid.

Preferably, the formulation is substantially free of particles visible to the naked human eye, as determined and characterized according to U.S. Pharmacopeial Convention (USP) Chapter 790 “VISIBLE PARTICULATES IN INJECTIONS” guidance of 2014 regarding parenteral medical products being “essentially free” of visible particulate matter (USP 790), which is incorporated herein by reference. Visible particles may be a sign of aggregation, agglomeration, and/or degradation of AAV particles, such that a lack of visible particles may be advantageous to indicate a particularly stable formulation free from substantial aggregation or agglomeration. Preferably, the formulation also comprises limited subvisible particles, as determined and characterized according to U.S. Pharmacopeial Convention (USP) Chapter 787 “SUBVISIBLE PARTICULATE MATTER IN THERAPEUTIC PROTEIN INJECTIONS” (USP 787), which is incorporated herein by reference. For example, the number of particles having a diameter of ≥25 μm is no more than 600 per vial, and the number of particles having a diameter of ≥10 μm is no more than 6000 per vial, in the formulation.

Optionally, the formulation has a Dv90 of less than 50 μm, preferably less than 25 μm, more preferably less than 10 μm. The Dv90 values represent the percent (90% in this case) of the formulation that has a size no larger than the specified value.

In some embodiments, the formulation of the invention may be used in treating Huntington's disease. Preferably, the formulation may be administered to a patient's brain by intrastriatal (corpus striatum) injection. For example, the formulation may be administer to a patient's brain by intrastriatal injection targeting the putamen and caudate nucleus, preferably using a micro-catheter, preferably using convection-enhanced delivery. Thus, in some specific embodiments, the formulation is administered to a patient's brain by intrastriatal injection targeting the putamen and caudate nucleus.

In some embodiments, the formulation may be administered by at least 3 injections per hemisphere of the brain. The 3 injections per hemisphere may be done at the same site, or at different sites within the hemisphere. For example, the at least 3 injection sites per hemisphere may include 2 in the putamen and 1 in the caudate of that hemisphere. In one particular example of the present invention, the formulation of the present invention is administered to a patient's brain by intrastriatal injection at 3 injection sites (2 in the putamen and 1 in the caudate) per hemisphere. Other numbers of injections, or combinations of injection sites, may also be used, depending on the particulars of the chosen administration route.

Optionally, the formulation of the present invention is administered at a dosage regime from 1E12 to 1E14 genome copies per patient, for example from 6E12 to 6E13 genome copies per patient. For example, when the formulation is administered to 3 injection sites per hemisphere, the injection volume may be 500 μL per injection site at a concentration (of the adeno-associated viral vector) of 2E12 or 2E13 gc/ml. That way the formulation is administered at a dosage of 6E12 or 6E13 genome copies per patient.

In some embodiments, the formulation of the invention may be administered together with a contrast agent visualizable by magnetic resonance imaging (MRI).

Alternatively, or in addition as another product of interest, the nucleotide sequence comprising the transgene encoding a microRNA as defined herein above may further comprise a nucleotide sequence encoding a polypeptide that serves as a selection marker protein to assess cell transformation and expression. Suitable marker proteins for this purpose are e.g. the fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene. Sources for obtaining these marker genes and methods for their use are provided in Sambrook and Russel, see below.

Furthermore, the nucleotide sequence comprising the transgene as defined herein above may comprise a further nucleotide sequence encoding a polypeptide that may serve as a fail-safe mechanism that allows to cure a subject from cells transduced with an AAV vector of the invention, if deemed necessary. Such a nucleotide sequence, often referred to as a suicide gene, encodes a protein that is capable of converting a prodrug into a toxic substance that is capable of killing the transgenic cells in which the protein is expressed. Suitable examples of such suicide genes include e.g. the E. coli cytosine deaminase gene or one of the thymidine kinase genes from Herpes Simplex Virus, Cytomegalovirus and Varicella-Zoster virus, in which case ganciclovir may be used as prodrug to kill the transgenic cells in the subject (see e.g. Clair et al., 1987, Antimicrob. Agents Chemother. 31: 844-849).

In one aspect the invention provides a method for preparing a drug product for administration to the central nervous system, the method comprising the steps of:
i) providing a formulation according to the invention; and
ii) aliquoting the formulation into a suitable dosage form.

The prepared drug product is suitable for administration to the central nervous system, for instance as described elsewhere herein. Accordingly, the method is suitable for preparing formulations for use according to the invention, preferably wherein the composition is for by intrastriatal administration.

In yet another aspect of the invention, the present invention provides a method of treating Huntington's disease by administering the formulation of the invention to a patient's brain.

### Definitions

In this document and in its claims, the verb “to comprise” and its conjugations is used in its non-limiting sense to mean that items following the word are included, but items not specifically mentioned are not excluded. In addition, the verb “to consist” may be replaced by “to consist essentially of” meaning that a combination or a composition as defined herein may comprise additional component(s) than the ones specifically identified, said additional component(s) not altering the unique characteristic of the invention. In addition, reference to an element by the indefinite article “a” or “an” does not exclude the possibility that more than one of the element is present, unless the context clearly requires that there be one and only one of the elements. The indefinite article “a” or “an” thus usually means “at least one”. Whenever a parameter of a substance is discussed in the context of this invention, it is assumed that unless otherwise specified, the parameter is determined, measured, or manifested under physiological conditions. Physiological conditions are known to a person skilled in the art, and comprise aqueous solvent systems, atmospheric pressure, pH-values between 6 and 8, a temperature ranging from room temperature to about 37° C. (from about 20° C. to about 40° C.), and a suitable concentration of buffer salts or other components.

In the context of this invention, a decrease or increase of a parameter to be assessed means a change of at least 5% of the value corresponding to that parameter. More preferably, a decrease or increase of the value means a change of at least 10%, even more preferably at least 20%, at least 30%, at least 40%, at least 50%, at least 70%, at least 90%, or 100%. In this latter case, it can be the case that there is no longer a detectable value associated with the parameter.

The word “about” or “approximately” when used in association with a numerical value (e.g. about 10) preferably means that the value may be the given value (of 10) more or less 10% of the value, optionally more or less 5%.

Each embodiment as identified herein may be combined together unless otherwise indicated. The invention has been described above with reference to a number of embodiments. A skilled person could envision trivial variations for some elements of the embodiments. These are included in the scope of protection as defined in the appended claims. All patent and literature references cited are hereby incorporated by reference in their entirety.

In addition, the following terms are used herein and defined as follows.
“AAV”—Adeno Associated Virus.
“Capsid”—A protein shell surrounding viral DNA, RNA, or microRNA that helps target the genetic material to specific cell types.
“Gene cassette”—A small piece of DNA, RNA, or microRNA containing the therapeutic gene and instructions for the cell on how to use the gene.
“Vector”—This may refer to the DNA, RNA, or microRNA molecule itself or the carrier construct containing the DNA, RNA, or microRNA to be delivered to a cell.
“microRNA” (also termed “miRNA” or “siRNA”)—a small single-stranded non-coding RNA molecule, typically containing a guide strand as described above.
“CSF”—human cerebrospinal fluid.
“HPBCD”—2-Hydroxypropyl-β-cyclodextrin.
“iPSCs”—induced pluripotent stem cells.
“ITR”—inverted terminal repeat.
“MRI”—magnetic resonance imaging.
“substantially”—the term “substantially” as used herein is a broad term, and is to be given its ordinary and customary meaning to a person of ordinary skill in the art (and is not to be limited to a special or customized meaning), and refers without limitation to being largely but not necessarily wholly that which is specified.

Any feature that has been described above in relation to any one aspect or embodiment of the invention is also disclosed hereby in relation to all other aspects and embodiments. Likewise, all combinations of two or more of the individual features or elements described above may be present in any aspect or embodiment. For brevity, all possible features and combinations have not been recited in relation to all aspects and embodiments, but they are expressly contemplated and hereby disclosed.

Embodiments of the present invention are described in the following paragraphs.

1. An isotonic pharmaceutical formulation comprising:
a buffer;
an adeno-associated viral vector with a transgene encoding a microRNA; and
a cyclodextrin or a derivative thereof.

2. The isotonic formulation of paragraph 1, wherein the formulation has an osmolarity of from 250 to 330 mOsm/kg, preferably from 270 to 310 mOsm/kg, for example from 280 to 300 mOsm/kg.

3. The isotonic formulation of paragraph 1 or 2, wherein the formulation has a pH value of from 6.5 to 8, preferably from 7 to 8, more preferably from 7.3 to 7.7, for example, 7.5.

4. The isotonic formulation of any one of the preceding paragraphs, wherein the formulation is essentially free of visible particles.

5. The isotonic formulation of any one of the preceding paragraphs, wherein the adeno-associated viral vector with a transgene encoding a microRNA targets huntingtin mRNA, for example, the microRNA targets exon1 of huntingtin mRNA.

6. The isotonic formulation of any one of the preceding paragraphs, wherein the adeno-associated viral vector comprises an AAV2 serotype, an AAV5 serotype, an AAV9 serotype, a hybrid AAV serotype, or a combination thereof, preferably the adeno-associated viral vector comprises an AAV5 serotype.

7 The isotonic formulation of any one of the preceding paragraphs, wherein the concentration of the adeno-associated viral vector is up to 5E13 gc/ml, preferably up to 2E13 gc/ml, for example, 2E12 gc/ml or 2E13 gc/ml.

8. The isotonic formulation of any one of the preceding paragraphs, wherein the cyclodextrin is an unsubstituted or substituted β-cyclodextrins, for example, 2-hydroxypropyl-β-cyclodextrin (HP—β-CD).

9. The isotonic formulation of any one of the preceding paragraphs, wherein the cyclodextrin is present in an amount less than about 4% w/v, preferably less than about 2% w/v, preferably about 0.05% w/v to about 4% w/v, more preferably about 0.05% w/v to about 2% w/v; for example, the cyclodextrin is 2-hydroxypropyl-β-cyclodextrin (HP-β-CD) present in an amount of about 0.1% w/v.

10. The isotonic formulation of any one of the preceding paragraphs, further comprising a sugar or sugar alcohol, preferably a monosaccharide, disaccharide, or sugar alcohol.

11. The isotonic formulation of paragraph 10, wherein the sugar or sugar alcohol is selected from the group consisting of trehalose, sucrose, maltose, mannitol, and derivatives and combinations thereof.

12. The isotonic formulation of paragraph 10 or 11, wherein the sugar or sugar alcohol is present in an amount of about 0.01% w/v to about 4% w/v, preferably about 0.05% w/v to about 2% w/V, preferably about 0.1% w/v to about 1% w/v, for example, 0.1% w/v.

13. The isotonic formulation of any one of paragraphs 10 to 12, wherein the sugar or sugar alcohol is mannitol and wherein mannitol is present in an amount of about 0.05% w/v to about 2% w/v.

14. The isotonic formulation of any one of the preceding paragraphs, further comprising a pharmaceutically acceptable salt, preferably a periodic group 1 or group 2 metal salt, for example, a salt selected from the group consisting of NaCl, KCl, CaCl₂), MgCl₂, and combinations thereof.

15. The isotonic formulation of paragraph 13, wherein the concentration of pharmaceutically acceptable salt is about 75 mM or higher, preferably about 75 mM to about 200 mM, more preferably about 75 mM to about 150 mM, for example about 135 mM.

16. The isotonic formulation of paragraph 14 or 15, wherein the salt is NaCl present in an amount of about 135 mM.

17. The isotonic formulation of any one of the preceding paragraphs, wherein the buffer comprises:
a Tris buffer, for example, a Tris buffer at a pH of about 7.5 to about 8.0; or
a phosphate buffer, for example, a phosphate buffer at a pH of about 7.0 to about 7.5; or
a citrate buffer, for example, a citrate buffer at a pH of about 5.5 to about 6.5;
for example, the buffer is a 20 mM Tris buffer at a pH of 7.5.

18. The isotonic formulation of any one of the preceding paragraphs, further comprising an amino acid

19. The isotonic formulation of paragraph 18, wherein the amino acid is selected from the group consisting of cysteine, arginine, histidine, glycine, and derivatives and combinations thereof, preferably, the amino acid comprises histidine and/or glycine.

20. The isotonic formulation of paragraph 19, wherein the amino acid is present at a concentration of about 2 mM to about 3 mM, preferably about 2.5 mM.

21. The isotonic formulation of any one of the preceding paragraphs, wherein the formulation is free of surfactant such as Polysorbate 20, Polysorbate 80, or Poloxamer 188.

22. The isotonic formulation of any one of the preceding paragraphs, wherein the formulation remains stable at room temperature (15° C.-25° C.) without substantial formation of aggregates or agglomerates for at least 12 hours, preferably at least 18 hours, for example at least 24 hours, after thawing.

23. The isotonic formulation of any one of the preceding paragraphs, wherein the formulation remains stable at a storage temperature of ≤−65° C. without substantial formation of aggregates or agglomerates for at least about 6 months, preferably at least about 12 months, more preferably at least about 24 months, for example at least 36 months.

24. The isotonic formulation of any one of the preceding paragraphs for use in treating Huntington's disease.

25. The isotonic formulation for use of paragraph 24, wherein the formulation is administered to a patient's brain by intrastriatal injection targeting the putamen and caudate nucleus, preferably using a micro-catheter, preferably using convection-enhanced delivery.

26. The isotonic formulation for use of paragraph 24 or 25, wherein the formulation is administered by 3 injections per hemisphere of the brain, for example, 2 to the putamen and 1 to the caudate per hemisphere.

27. The isotonic formulation for use of any of paragraphs 24 to 26, wherein the formulation is administered together with a contrast agent visualisable by MRI.

28. The isotonic formulation for use of any of paragraphs 24 to 27, wherein the formulation is administered at a dosage of 6E12 or 6E13 genome copies per patient.

29. A method of treating Huntington's disease by administering the isotonic formulation of any one of the preceding paragraphs to a patient's brain.

### FIGURES

Specific embodiments of the present invention will be described by way of example, with reference to the accompanying drawings in which:

FIG. 1 shows T=0 Protein Content as followed by Absorbance at 280 nm (corrected for turbidity and path length in three multi-well plates; arbitrary units). A: F/T at −80° C.; B: 28° C. and C: 40° C.

FIG. 2 shows T=0 Fluorescence as followed by Fluorescence at 335 nm (bottom measurement from three multi-well plates; arbitrary units). A: F/T at −80° C.; B: 28° C. and C: 40° C.

FIG. 3 shows 280 nm absorbance and 335 nm fluorescence measurements as a function of the accelerated stress conditions and duration for the different salt concentrations. The different pHs were pooled.

FIG. 4 shows 280 nm absorbance and 335 nm fluorescence measurements as a function of the accelerated stress conditions and duration. The different pHs are shown as a gradient from dark (pH 8.0) to light (pH 5.0). The different salt concentrations were pooled.

FIG. 5 shows T=0 protein content as followed by Absorbance at 280 nm (corrected for turbidity and path length in four multi-well plates; arbitrary units). A: F/T at −80° C.; B: 28° C.; C: 40° C. and D: Shaking.

FIG. 6 shows T=0 fluorescence as followed by Fluorescence at 335 nm (bottom measurement from four multi-well plates; arbitrary units). A: F/T at −80° C.; B: 28° C.; C: 40° C. and D: Shaking.

FIG. 7 shows T=0 protein content as followed by Absorbance at 280 nm (corrected for turbidity and path length in four multi-well plates; arbitrary units). A: F/T at −80° C.; B: 28° C.; C: 40° C. and D: Shaking.

FIG. 8 shows T=0 Fluorescence as followed by Fluorescence at 335 nm (bottom measurement from four multi-well plates; arbitrary units). A: F/T at −80° C.; B: 28° C.; C: 40° C. and D: Shaking.

FIG. 9 shows T=0 protein content as followed by Absorbance at 280 nm (corrected for turbidity and path length in four multi-well plates; arbitrary units). A: F/T at −80° C.; B: 28° C.; C: 40° C. and D: Shaking. The scale is greener the higher the value according to an Excel algorithm.

FIG. 10 shows T=0 fluorescence as followed by Fluorescence at 335 nm (bottom measurement from four multi-well plates; arbitrary units). A: F/T at −80° C.; B: 28° C.; C: 40° C. and D: Shaking.

FIG. 11 shows protein content as followed by 280 nm absorbance after stresses at 28° C. and 40° C. for 0, 3, or 7 days; F/T for 2 and 5 cycles, and 2 hours of shaking. Different shades of grey represent different mannitol concentrations, while the different columns the different HPBCD concentrations.

FIG. 12 shows formulation confirmation fluorescence measurements after stresses at 28° C. and 40° C. for 0, 3, or 7 days; F/T for 2 and 5 cycles, and 2 hours of shaking. Different shades of grey represent different mannitol concentrations, while the different columns the different HPBCD concentrations.

FIG. 13 shows formulation confirmation turbidity. Protein degradation is followed by 350 nm turbidity after stresses at 28° C. and 40° C. for 0, 3, or 7 days; F/T for 2 and 5 cycles and 2 hours of shaking. Different shades of grey represent different mannitol concentrations, while the different columns the different HPBCD concentrations.

FIG. 14 shows the effect of excipients on iPSC-derived neurons: cells were challenged with 0.1% w/v mannitol+0.1% w/v HPBCD, 2% w/v HPBCD and PBS/sucrose, as a control, and observed under the microscope after 14 hours.

### EXAMPLES

### Materials

The following materials were used in the examples and are available as specified below in Table 1.

TABLE 1
Materials used in the examples and their suppliers

| Material(s) | Supplier |
|---|---|
| Acetate (Acetic acid) | Acetic acid, Merck |
| Citric acid | Merck |
| Glycine | Merck |
| HPBCD | Sigma Aldrich |
| L-Histidine | Sigma |
| Maltose (D-(+)- Maltose monohydrate) | Sigma |
| Mannitol (D-Mannitol) | Sigma |
| MgCl₂ | Sigma |
| NaCl (solid) | Sigma Aldrich |
| Sodium phosphate dibasic | Merck |
| Sucrose | Merck |
| Trehalose | Sigma |
| Tris | Merck |

### Data Points

The stress conditions and time points for data collection for each Example are as specified below in Table 2.

TABLE 2
Stress Conditions and Data Collection Time Points for the Examples

| | Example 1 | | Example 2 | | Example 3 | |
|---|---|---|---|---|---|---|
| Initial T = 0 | Done | | Done | | Done | |
| measurements | | | | | | |
| Storage at 28° C. | 2 and 7 | days | 3 | days | 2 and 7 | days |
| Storage at 40° C. | 2 and 7 | days | 3 | days | 2 and 7 | days |
| Freeze at −80° C. | 1, 3, and 10 | cycles | 1 and 3 | cycles | 2 and 5 | cycles |
| (atleast 1 h) and | | | | | | |
| thaw at RT* (1 hour) | | | | | | |
| Shaking at | N.A.** | | 4 | hours | 2 | hours |
| maximum vortex | | | | | | |
| velocity at RT | | | | | | |

*RT: room temperature
**N.A.: not available

### Measurements

1. The read-outs obtained were as follows:
   Absorbance at 260 nm, which correlates with DNA concentration.
   Absorbance at 280 nm, which correlates with protein concentration.
   Absorbance at 350 nm (turbidity), which correlates with protein aggregation, or agglomeration and/or particle formation.
   Absorbance at 900 nm, which correlates with the background signal from the plastic of the wells and seal.
   Absorbance at 975 nm measures the water absorbance, thus, the path length.
   Tryptophan fluorescence at 280/335 nm, measured from the top and the bottom of the micro-well plate. This measurement correlates with protein and/or capsid unfolding and denaturation. The bottom read-out was used in the figures.
2. Visual Inspection

Visual inspection was carried out as generally accepted.
3. Data Processing

The protein and DNA concentrations were obtained as follows: According to the Lambert-Beer law, the absorbance is proportional to the concentration and the light path length. A constant named the extinction coefficient turns this into an equation A=cc·L·ε, where A is the absorbance, cc is the concentration, L is the light path length and & is the extinction coefficient. The light path length is measured by the water absorbance measured at 975 nm (A975) minus the plastic background measured at 900 nm (A900) thus: L=A975-A900

The protein concentration is measured by measuring the absorbance at 280 nm (A280), thus: A280=_{CC(protein)·L·ε(protein)}. The background from the precipitated and aggregated proteins or turbidity is measured by the absorbance at 350 nm (A350). It is assumed to be similar to the contribution of turbidity at 280 nm, and thus, deducted from the absorbance at 280 nm. Then:

$${cc}_{( protein )} \cdot {ε}_{( protein )} = ( {A}_{280} - {A}_{350} ) / ( {A}_{975} - {A}_{900} )$$

Since ε (protein) is a constant, we can conclude that cc(protein)·ε(protein), is proportional to the protein concentration, which is computed from the different absorbance values measured (A₂₈₀, A₃₅₀, A₉₇₅ & A₉₀₀).

For the DNA concentration at 260 nm, a similar reasoning was applied.

### Example 1: Pre-Formulation

The initial solution used in Example 1 was rAAV5 with transgene miHTT targeting exon 1 of mutant huntingtin mRNA (4E13 gc/mL) in PBS−/− (NaCl 137 mM; KCl 2.7 mM; Na₂HPO4 10 mM and KH₂PO₄ 1.8 mM), 5% w/v sucrose.

The initial solution containing rAAV5-miHTTs was buffer exchanged in a multi-well system into thirty different formulations comprising different pHs, buffer types, and salt contents using 96 well filter plates (100 kDa cut-off). After the buffer was exchanged and filtered through a 0.2 μm filter, the formulations were aliquoted into the plates according to the plate layout and buffer formulation compositions as specified in Table 3 below. Each formulation is prepared in duplicate. The outer rows and columns are filled with WFI to rule out plate location effects. The buffers have overlapping pHs, to differentiate between a buffer and a pH effect.

TABLE 3
Buffer Plate Layout and Buffer Formulation Compositions for Example 1

| | | | Acetate | | Citrate | | | Phosphate | | | Tris | | |
|---|---|---|---|---|---|---|---|---|---|---|---|---|---|
| | | | pH | | | | | | | | | | |
| | | WFI | 5.0 | 5.5 | 5.5 | 6.0 | 6.5 | 6.5 | 7.0 | 7.5 | 7.5 | 8.0 | WFI |
| Salt | Buffer | 1 | 2 | 3 | 4 | 5 | 6 | 7 | 8 | 9 | 10 | 11 | 12 |
| WFI | A | | | | | | | | | | | | |
| WFI | B | | | | | | | | | | | | |
| | 5 | | | | | | | | | | | | |
| 0.075M NaCl | D | | | | | | | | | | | | |
| | E | | | | | | | | | | | | |
| 0.15 mM NaCl | F | | | | | | | | | | | | |
| | G | | | | | | | | | | | | |
| WFI | H | | | | | | | | | | | | |

The different preparations were monitored for absorbance (at 280 nm), fluorescence (at 335 nm), and turbidity (at 350 nm). Corrections in the absorbance for background due to turbidity were carried out, as well as corrections for the path length. Three plates were prepared per each stress condition as specified above in Table 2 above, and each condition (or each well) was measured in duplicate and the duplicate values were averaged.

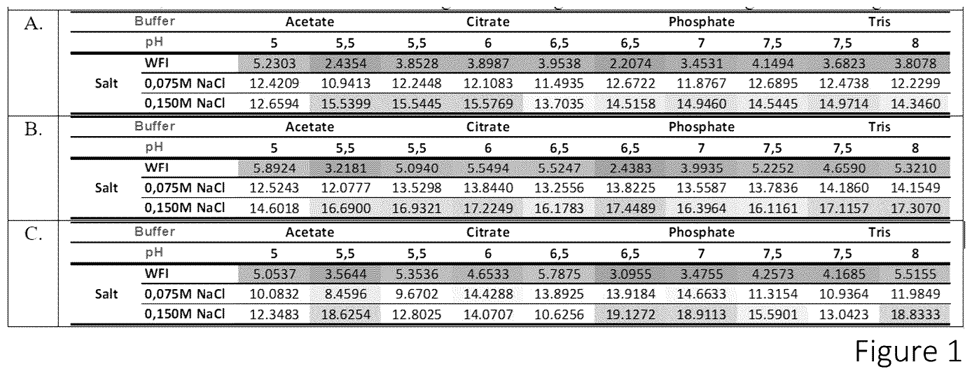

A tabular representation of the results obtained at T=0 is shown in FIG. 1. FIG. 1 clearly shows that the three multi-well plates have similar values for the corrected absorbance at 280 nm (protein concentration) as expected. In addition, for the three plates at T=0, the absorbances are higher in the presence of salt, particularly at 150 mM NaCl, therefore showing a higher protein content and a lower degree of aggregation or agglomeration. This can be concluded since the values measured include the correction for the background as explained in the previous section. All buffers could be potentially stabilizing buffers, therefore, showing a positive effect for the presence of NaCl. Buffers with a pH of 6.5 to 8 (in particular with the addition of salt) are preferable as higher absorbance (protein concentration) are observed.

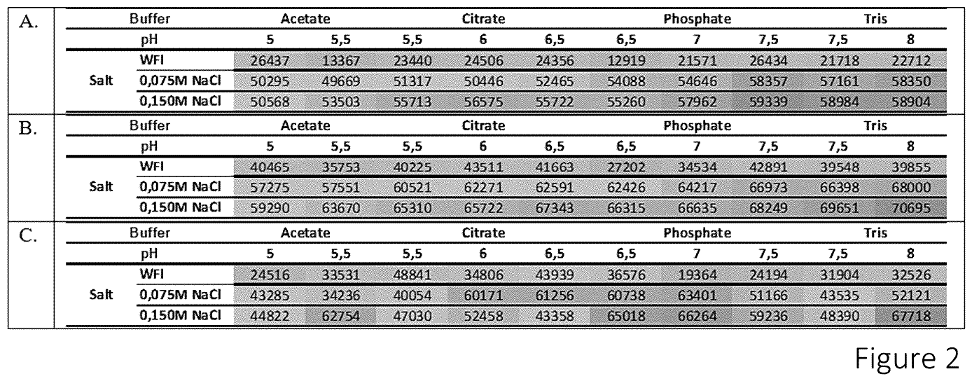

The results obtained from the fluorescence measurements (protein and/or capsid unfolding and denaturation) are shown in FIG. 2. The lower the fluorescence, the more unfolded the proteins are. These results suggest that the buffers which best stabilize the virus capsids are citrate at pH 6.5, phosphate at pH; 7.0 and 7.5, and Tris at pH 7.5 and 8.0 with 75 mM and 150 mM NaCl, with the later NaCl concentration seeming to be improved over the former.

Accelerated studies were carried out for a partial battery of conditions. The various assays were followed after two and seven days for the samples at 28° C. and 40° C.; and after 1, 3, and 10 cycles for the samples under F/T stress.

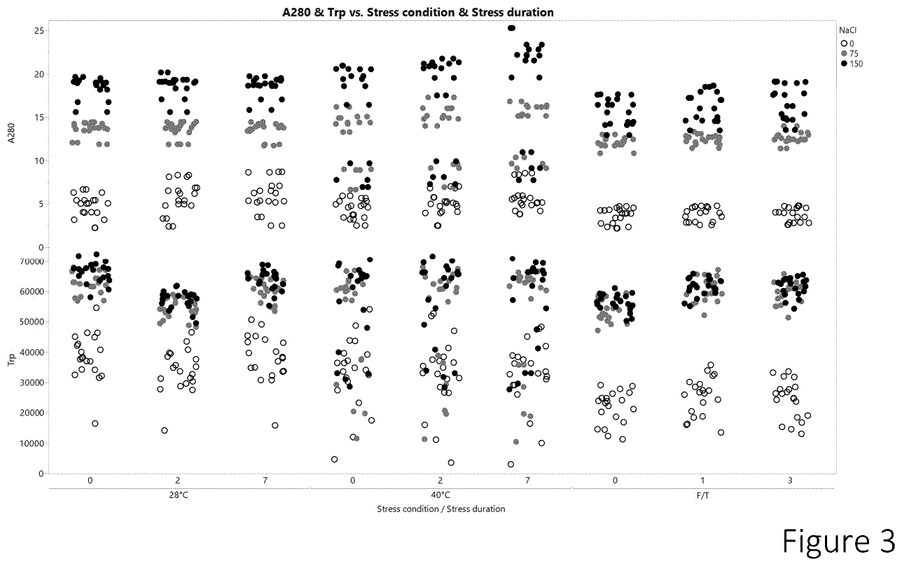

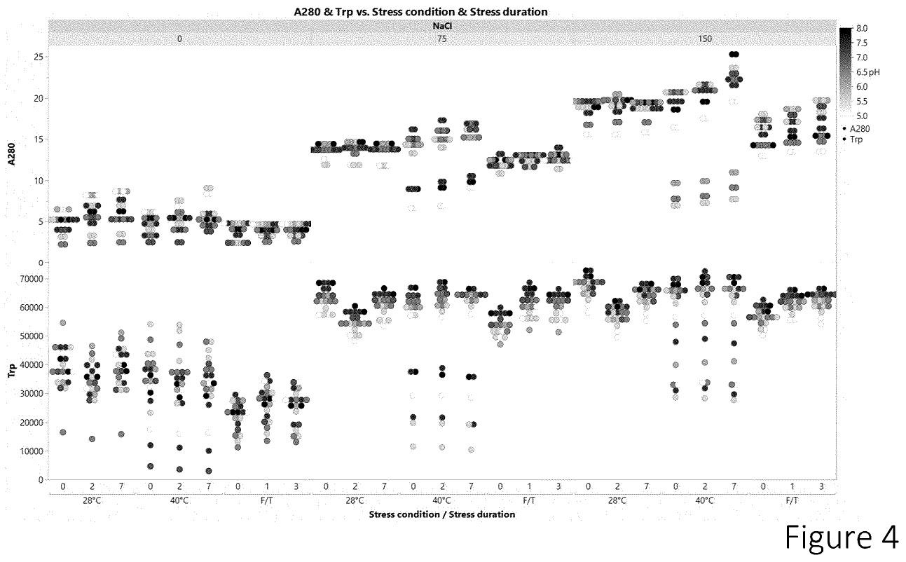

FIG. 3 shows the 280 nm absorbance and the 335 nm fluorescence (Trp) measured in the accelerated stress studies for the different formulations. It is seen that the higher the salt concentration, the higher the 280 nm absorbance and the 335 nm fluorescence. This observation, which is the same as observed for T=0, shows that higher salt concentrations are beneficial for the formulation in terms of protein concentration. In FIG. 4, the 280 nm absorbance and the 335 nm fluorescence are shown as a function of the different stresses applied. In this graph, there are three panels for the different salt concentrations: 0 mM left panel, 75 mM middle panel, and 150 mM right panel. As in the previous figure, the observed effect of the salt on the absorbance and fluorescence is very clear: the higher the salt concentration, the higher the absorbance and fluorescence. Showing once more the favourable effect of salt. The effect of the pH is, also, clearly seen: the higher the pH, the higher the absorbance and the fluorescence. This effect is especially observed for the fluorescence (Trp). Its magnitude is lower than the one observed for the salt effect.

It is therefore concluded from Example 1 that the salt concentration (above 75 mM) and the pH (above 7.0) have a beneficial effect on enhancing the stability of the capsids. Tris pH 7.5 and 8.0, and phosphate pH7.0 buffers were chosen for further tests and optimizations.

### Example 2: Excipient Screening

The drug product used in Example 2 was rAAV5 with transgene miHTT targeting exon 1 of mutant huntingtin mRNA (concentration of rAAV5 was 2E13 gc/mL).

In the first excipient screening, rAAV5-miHTTs was buffer exchanged with different buffer formulations. The formulations were and filtered through a 0.2 μm filter and aliquoted into the plates according to the plate layout and buffer formulation compositions as specified in Table 4 below. Each formulation is prepared in duplicate. Several multi-well plates were prepared and assigned to different stress conditions as specified in Table 2 above. The outer rows and columns are filled with WFI to rule out plate location effects. The buffers (phosphate or Tris) have different pHs and contain increasing amounts of select excipients, such as cysteine, arginine, and MgCl₂

TABLE 4
Buffer Plate Layout and Buffer Formulation Compositions for Example 2 (1)

| | | | Cysteine | | | | Arginine | | | MgCl₂ | | | |
|---|---|---|---|---|---|---|---|---|---|---|---|---|---|
| | | | 0 | 1 | 2.5 | 5 | 1 | 2.5 | 5 | 75 | 150 | | |
| | | WFI | mM | mM | mM | mM | mM | mM | mM | mM | mM | WFI | WFI |
| | | Buffer | | | | | | | | | | | |
| pH | | 1 | 2 | 3 | 4 | 5 | 6 | 7 | 8 | 9 | 10 | 11 | 12 |
| WFI | A | | | | | | | | | | | | |
| pH 7.0 | B | | | | | | | | | | | | |
| | C | | | | | | | | | | | | |
| pH 7.5 | D | | | | | | | | | | | | |
| | E | | | | | | | | | | | | |
| pH 8.0 | F | | | | | | | | | | | | |
| | G | | | | | | | | | | | | |
| WFI | H | | | | | | | | | | | | |

The different preparations were monitored for absorbance (at 280 nm), fluorescence (at 280/335 nm), and turbidity (at 350 nm). Corrections in the absorbance for background due to turbidity were carried out, as well as corrections for the path length. One plate was prepared per each stress condition as specified above in Table 2, and each condition (or each well) was measured in duplicate and duplicate values were averaged. The plates included a control sample without any excipient for each of the different buffers and pHs: Phosphate pH 7.0, Tris pH 7.5 and Tris pH 8.0.

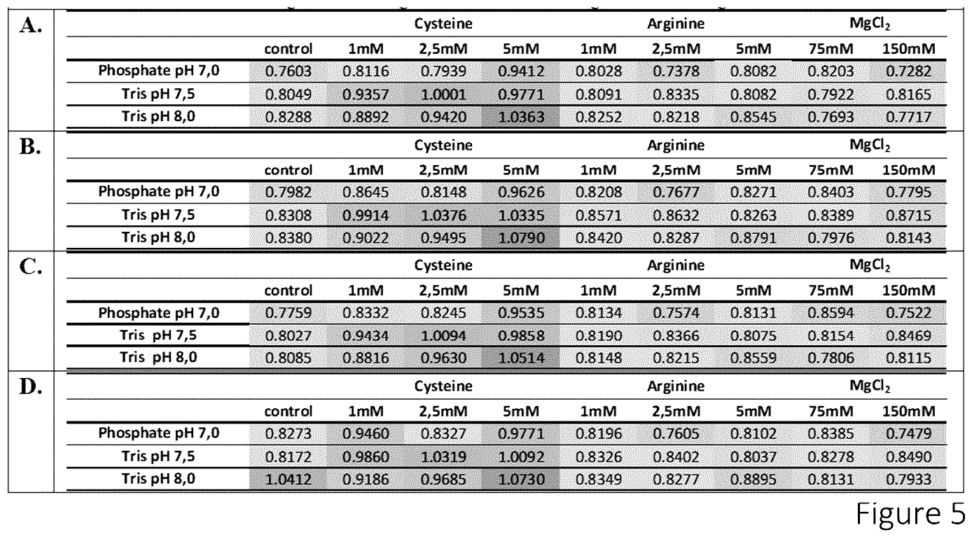

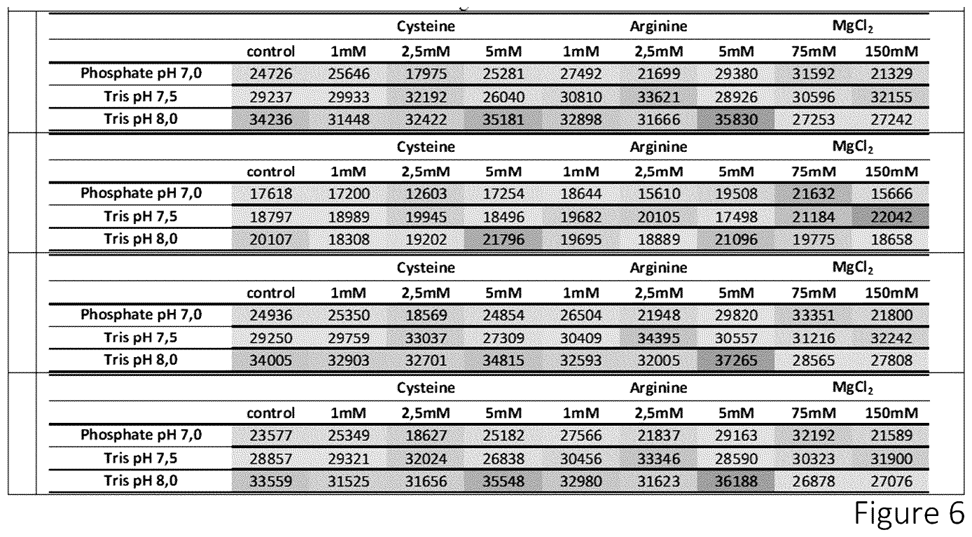

A tabular representation of the results obtained at T=0 is shown in FIGS. 5 and 6.

FIG. 5 clearly shows that the four multi-well plates have similar values for the corrected absorbance at 280 nm (protein concentration) as expected. Except for the Tris pH 8.0 control in the T=0 plate for shaking (FIG. 5, D). In addition, for the four plates at T=0, the absorbances are higher in the presence of cysteine, therefore showing a higher protein content and a lower degree of aggregation or agglomeration. Among the cysteine-containing formulations, those at pH 7.5 show a more significant stabilizing effect. This can be concluded since the value measured include the correction for the background as explained in the previous section.

The results obtained from the fluorescence measurements (protein and/or capsid unfolding and denaturation) are shown in FIG. 6. The lower the fluorescence, the more unfolded the proteins are. These results suggest that the buffers which best stabilize the virus capsids are either Tris at pH 7.5 with 2.5 mM of cysteine or arginine or Tris pH 8.0 with or without 5 mM of cysteine or arginine.

Accelerated studies were carried out for a partial battery of conditions. The various assays were followed after one and two days for the samples at 28° C. and 40° C.; after 1 and 3 cycles for the samples under F/T stress and after 1 hour of shaking at RT. The different excipients: cysteine, arginine and MgCl₂ do not appear to have an obvious or measurable effect on the stability of the product when compared to the control conditions (buffer alone) at any of the tested pHs: 7.0; 7.5 or 8.0 (data not shown).

A second excipients screening experiment was performed to assessed the effect that different sugars had on the stability of the drug product in the absence and presence of 2.5 mM glycine or 2.5 mM histidine. The pH was set to 7.5 in a Tris buffer to keep the formulation close to CSF pH (pH 7.3-7.4 for CSF).

In the second excipient screening, the formulation matrix tested is shown in Table 5. Several multi-well plates were prepared and assigned to different stress conditions as specified in Table 2 above. The buffer is Tris 20 mM pH 7.5 contains either 2.5 mM Glycine or 2.5 mM Histidine and a pallet of excipients at two final concentrations, 4% w/v, and 2% w/v, among them mannitol, maltose, trehalose, and 2-Hydroxypropyl-β-cyclodextrin (HPBCD). The combination of trehalose and HPBCD was also tested at 2% w/v and 1% w/v for each of the excipients.

TABLE 5
Buffer Plate Layout and Buffer Formulation Compositions for Example 2 (2)

| | | | 4% | 4% | 4% | 4% | 2% HPBCD + | 2% | 2% | 2% | 2% | 1% HPBCD + | |
|---|---|---|---|---|---|---|---|---|---|---|---|---|---|
| | | WFI | Mannitol | Maltose | HPBCD | Trehalose | 2% Trehalose | Mannitol | Maltose | HPBCD | Trehalose | 1% Trehalose | WFI |
| Buffer | | 1 | 2 | 3 | 4 | 5 | 6 | 7 | 8 | 9 | 10 | 11 | 12 |
| WFI | A | | | | | | | | | | | | |
| Tris | B | | | | | | | | | | | | |
| 20 mM | C | | | | | | | | | | | | |
| pH 7.5 | | | | | | | | | | | | | |
| Tris | D | | | | | | | | | | | | |
| 20 mM | E | | | | | | | | | | | | |
| pH 7.5 + | | | | | | | | | | | | | |
| 2.5 mM | | | | | | | | | | | | | |
| glycine | | | | | | | | | | | | | |
| Tris | F | | | | | | | | | | | | |
| 20 mM | G | | | | | | | | | | | | |
| pH 7.5 + | | | | | | | | | | | | | |
| 2.5 mM | | | | | | | | | | | | | |
| histidine | | | | | | | | | | | | | |
| WFI | H | | | | | | | | | | | | |

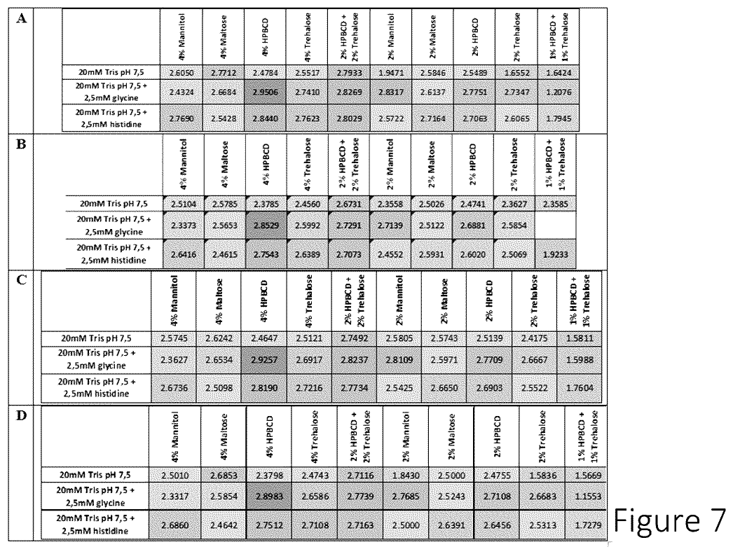

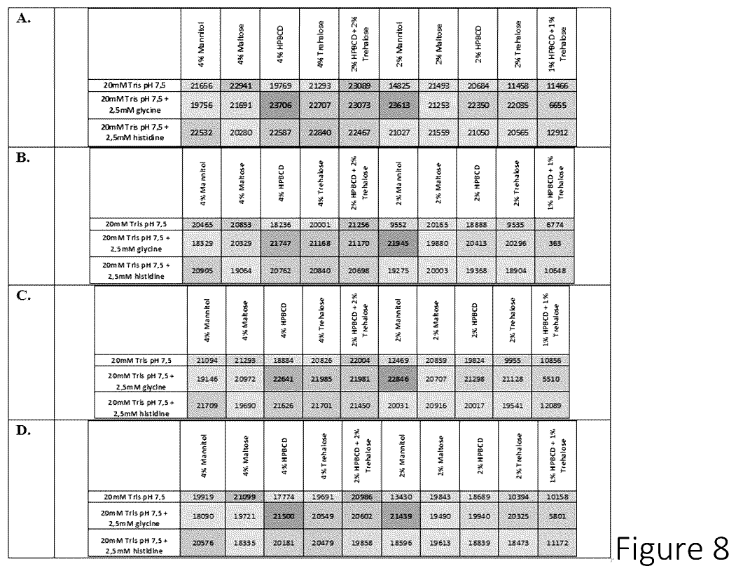

A tabular representation of the results obtained at T=0 is shown in FIGS. 7 and 8.

FIG. 7 shows the T=0 for the four micro-wells prepared for the different stresses: freeze/thaw, thermal stress at 28 and 40° C., and shaking. From the corrected 280 nm absorbance measurements, it is observed that higher osmolality formulations have a positive effect in comparison to those of a lower osmolality as seen by the formulations from the middle to the left side of the plate. HPBCD has a positive effect; either alone or in combination with 2% w/v trehalose. The presence of the amino acids glycine and histidine appeared to have an impact on the stability, both in a similar manner.

The fluorescence measurements shown in FIG. 8 are similar to those in FIG. 7, although a little more pronounced. The effects of 4% w/v HPBCD and 2% w/v mannitol as stabilizing excipients are observed for both buffers with glycine or histidine, stronger in the former than in the latter. The combination of HPBCD and trehalose also shows a stabilizing effect.

Accelerated studies were carried out for a partial battery of conditions. The various assays were followed after one and three days for the samples at 28° C. and 40° C.; after 1 and 3 cycles for the samples under F/T stress and after 4 hours of shaking at RT; The effect of the different stresses does not reveal any additional influence of the different sugars on the stability of the samples (data not shown).

### Example 3: Formulation Confirmation

To confirm the formulation, various buffer formulations were subject to further tests and optimizations. Several multi-well plates were prepared and assigned to different stress conditions as specified in Table 2 above. The basic buffer is a Tris 20 mM buffer pH 7.5 with 5 mM histidine, 2.5 mM MgCl₂, 2.5 mM KCl, 2.5 mM CaCl₂) and NaCl in sufficient amounts to reach the desired osmolality (290 mOsm/kg). The salts were added to mimic the composition of the CSF. The main variables of this Examples are the concentrations of mannitol and HPBCD on their own or in combinations. The concentration of mannitol and HPBCD ranged from low: 0.05% w/v to high: 4% w/v.

The drug product used in Example 3 was rAAV5 with transgene miHTT targeting exon 1 of mutant huntingtin mRNA.

The formulation matrix tested for Example 3 is shown in Table 6. Each formulation is prepared in triplicates. The outer rows and columns are filled with WFI to rule out plate location effects.

TABLE 6
Buffer Plate Layout and Buffer Formulation Compositions for Example 3

| Buffer | 1 | 2 | 3 | 4 | 5 | 6 |
|---|---|---|---|---|---|---|
| TH(+) buffer: | PBS pH | 0.05% | 0.1% | 0.5% | 1% | 2% |
| 20 mM Tris pH | 7 + 5% | Mannitol | Mannitol | Mannitol | Mannitol | Mannitol |
| 7.5 + 5 mM | sucrose | in TH(+) | in TH(+) | in TH(+) | in TH(+) | in TH(+) |
| histidine + | control | | | | | |
| 2.5 mM | Th(+) | 0.05% | 0.1% | 0.5% | 1% | 2% |
| MgSO4 + 2.5 | buffer | Mannitol + | Mannitol + | Mannitol + | Mannitol + | Mannitol + |
| mM CaCl2 + | control | 0.01% | 0.1% | 1% | 2% | 4% |
| 2.5 mM KCl + | | HPBCD | HPBCD | HPBCD | HPBCD | HPBCD |
| NaCl (for | | in TH(+) | in TH(+) | in TH(+) | in TH(+) | in TH(+) |
| isoosmolality) | | | | | | |

| Buffer | 7 | 8 | 9 | 10 | 11 | 12 | A |
|---|---|---|---|---|---|---|---|
| TH(+) buffer: | 0.01% | 0.1% | 1% | 2% | 4% | | B |
| 20 mM Tris pH | HPBCD | HPBCD | HPBCD | HPBCD | HPBCD | | C |
| 7.5 + 5 mM | in TH(+) | in TH(+) | in TH(+) | in TH(+) | in TH(+) | | D |
| histidine + | 2% | 1% | 0.5% | 0.1% | 0.05% | | E |
| 2.5 mM | Mannitol + | Mannitol + | Mannitol + | Mannitol + | Mannitol + | | F |
| MgSO4 + 2.5 | 0.01% | 0.1% | 1% | 2% | 4% | | G |
| mM CaCl2 + | HPBCD | HPBCD | HPBCD | HPBCD | HPBCD | | |
| 2.5 mM KCl + | in TH(+) | in TH(+) | in TH(+) | in TH(+) | in TH(+) | | |
| NaCl (for | | | | | | | H |
| isoosmolality) | | | | | | | |

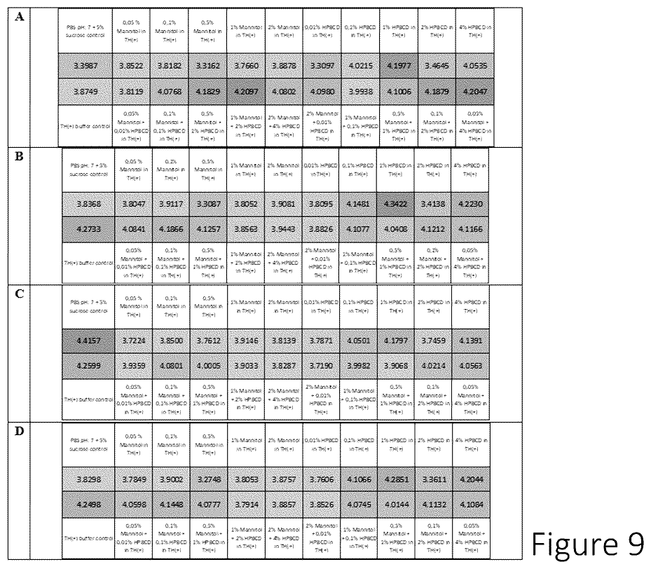

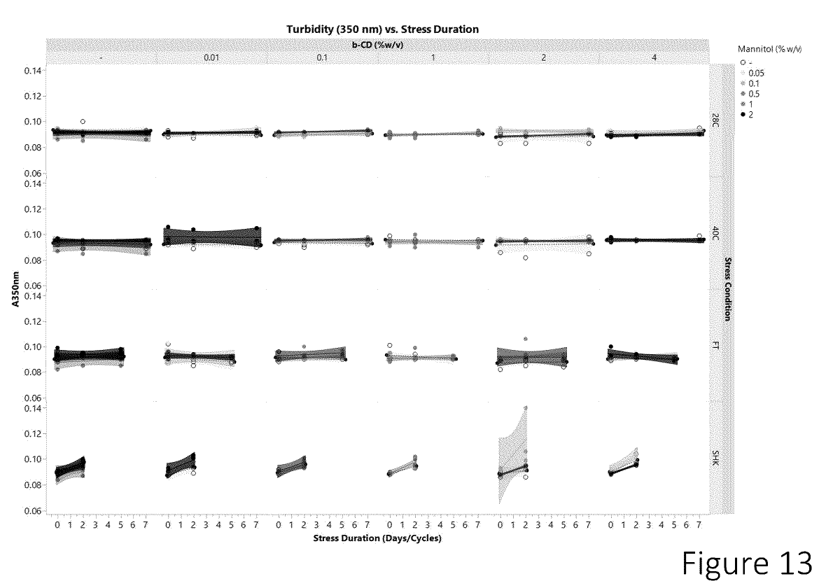

FIGS. 9 to 13 show the results of the formulation confirmation experiment.

FIG. 9 shows the 280 nm absorbance of the plates as measured at T=0. The variation of the different plates measured is higher than expected. In general terms, whenever two sugars are present, the effects appear more favourable than when only one sugar is present. The exceptions may be the formulations that contain HPBCD which seem to always have a better formulation profile.

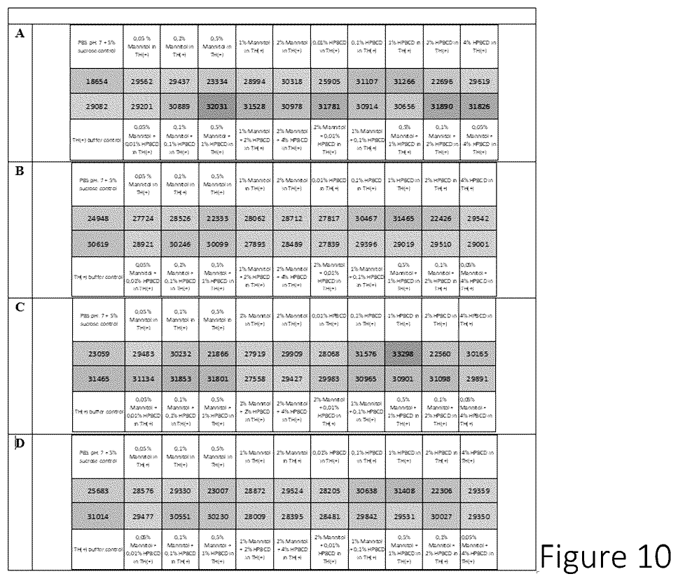

The fluorescence T=0 measurements (FIG. 10) show a similar trend as observed in the absorbance measurements (FIG. 9), where the combinations of mannitol and HPBCD showed a favourable effect on the stability of the samples when compared with both sugars on their own.

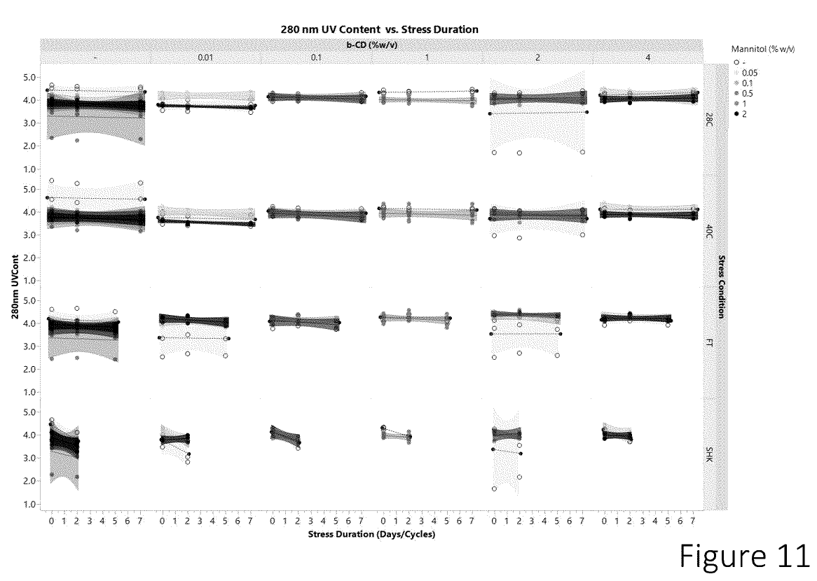

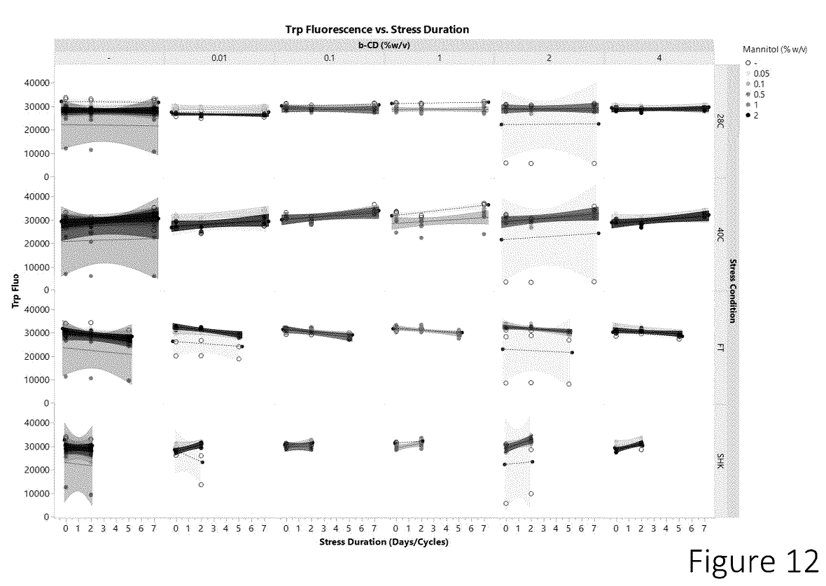

Several concentrations of HPBCD and mannitol alone and in combinations were tested to confirm the most suitable concentrations. FIG. 11 shows the changes in protein concentration as measured by 280 nm absorbance under different stress conditions and durations. The different mannitol and/or HPBCD concentrations show similar effects as seen by the overlapping curves for any of the different stresses. Similar observations can be concluded from the 350 nm turbidity measurements in FIG. 12. It is also seen that the stresses at 28° C. and 40° C. and the F/T do not manage to degrade the drug product further from the effect observed at T=0. Only the shaking stress manages to degrade the product further than in T=0 (FIG. 11 and FIG. 12 last row), but there are not any clear differences between the different formulations.

Similarly, it was observed that the different stresses do not manage to change the conformation of the capsids as demonstrated by the fluorescence measurements (data not shown).

The different mannitol or/and HPBCD concentrations do not show significant differences as stability-enhancing excipients beyond T=0 as seen in the figures. This may point out that the stabilizing effects of the different formulations achieved at T=0 are still valid under the stresses applied.

### Example 4: In Vitro Assessment

Since there were no differences between the tested formulations concerning the product stability, chosen formulations were tested in an in-vitro cellular assay using induced pluripotent stem cells (iPSCs) differentiated into neurons to represent the CNS cells.

In this Example, the iPSC-derived frontal brain-like neurons were seeded in pre-coated 24-wells plates. Coating was performed in two steps, first a coating with 1 mL of 0.1 mg/mL poly-D-lysine per well, which was incubated for 2 hours at 37° C. or overnight (~20 hours) at 2-8° C. After the first coating, the poly-D-lysine solution was aspirated from wells and wells were washed twice with 2 mL DPBS per well. The second coating was performed with 1 mL of 10 μg/mL laminin per well, and was incubated for 2 hours at 37° C. or overnight (~20 hours) at 2-8° C. After the second coating, cells were seeded in the 24-wells plates, and laminin solution was aspirated briefly before cells were seeded to ensure that wells were not dried out. The iPS-derived frontal brain-like neurons were always counted using the NucleoCounter NC-100 before seeding in 24-well plates. Cells were seeded in 1×105 cells/well in 0.5 mL supplemented neuron maturation medium from STEMCELL. After cells were seeded, medium was refreshed twice per week and washed once with 1 mL DPBS before adding the fresh medium. All solutions were pre-warmed at 37° C. before use.

Day 0 cells were seeded and allowed to grow for 2 days. On day 2 the cells were exposed to formulations 1, 2 and 3, as specified in Table 7 below. Fresh medium was added during each experiment on days of transduction.

TABLE 7
Formulations 1 to 3 for Example 4

| | Formulation 1 | Formulation 2 | Formulation 3 |
|---|---|---|---|
| Drug Product | AAV5-miHTT (an AAV vector with transgene encoding | | |
| | for miRNA that targets huntingtin mRNA) | | |
| Concentration | 1E12 per well | | |
| of Drug Product | | | |
| Buffer composition | PBS buffer pH 7.1 | Tris 20 mM pH 7.5, 5 mM Histidine, | |
| | (NaCl 137 mM; KCl 2.7 | 2.5 mM KCl, 2.5 mM CaCl2, 2.5 mM | |
| | mM; Na₂HPO₄ 10 mM | MgSO4, NaCl added to achieve | |
| | and KH₂PO₄ 1.8 mM) | required tonicity (290 | |
| Further Excipients | 5 w/v sucrose | HPBCD 0.1% w/v + | HPBCD 2% w/v |
| | | Mannitol 0.1% w/v | |

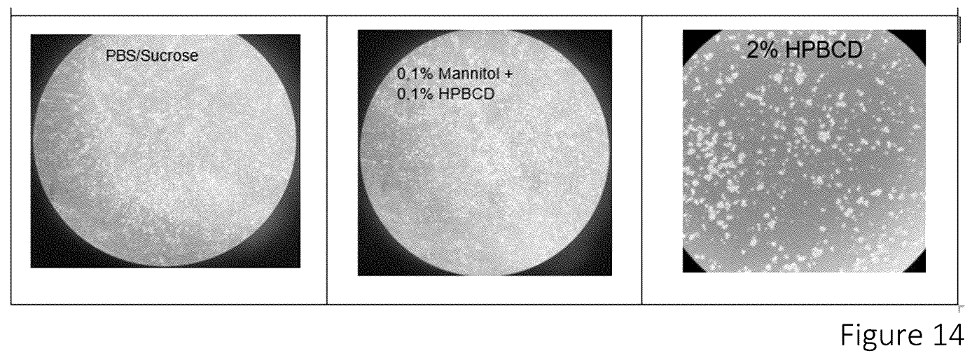

iPSCs-derived neuronal cells were treated with HPBCD and/or mannitol at different concentrations from 0.1% w/v up to 2% w/v. FIG. 14 shows the morphology of the cells as observed through the microscope. It is observed that the cells are especially viable in the medium containing 0.1% w/v HPBCD and 0.1% w/v mannitol or the control (PBS/sucrose), compared to 2% w/v HPBCD after a minimum of 1.5 hours.

### CONCLUSIONS

The conclusions of this study are as follows:
The stabilization effect of all tested formulations on the stability of the drug product measured by 280 nm absorbance, 350 nm turbidity, and 335 nm fluorescence is seen immediately at T=0.
Cyclodextrins, such as HPBCD, show a clear beneficial stabilizing effect.
Sugars and sugar alcohols, such as mannitol and trehalose, optionally combined with HPBCD, also have a stabilizing effect.
A salt, such as NaCl, also assists in stabilizing the drug product. Salt (e.g. NaCl) addition at concentration levels of 75 mM or higher is beneficial.
Neutral or slightly basic formulations pH (7.0-8.0) are better for the stability of the drug product.
Amino acids, such as glycine or histidine, show a stabilizing effect.
In-vitro experiments on a neuron cell model (iPSCs-derived neurons), show that 0.1% w/v HPBCD in combination with 0.1% mannitol does not interfere with the neuronal cell's viability. Therefore, a formulation containing 0.1% w/v mannitol and 0.1% w/v HPBCD in a slightly basic pH with the NaCl concentration adjusted to keep the solution isotonic (osmolality between 270 and 310 mOsm/kg for CSF) is preferred.

The following formulation of the invention is therefore specifically exemplified:
rAAV5 with transgene miHTT targeting exon 1 of mutant huntingtin mRNA (concentration of rAAV5 at 2E12 gc/mL or 2E13 gc/mL).
20 mM Tris at pH 7.5,
135 mM sodium chloride,
0.1% w/v mannitol,
0.1% w/v HP-β-cyclodextrin, and
2.5 mM L-Histidine.

The above embodiments have been described by way of example only. Many other embodiments falling within the scope of the accompanying claims will be apparent to the skilled reader. Therefore, although the present invention has been described in connection with the preferred embodiments of the various figures, it is to be understood that other similar embodiments may be used, or modifications or additions may be made to the described embodiment for performing the same function of the present invention without deviating therefrom. Therefore, the present invention should not be limited to any single embodiment, but rather construed in breadth and scope in accordance with the recitation of the appended claims.

1. An isotonic pharmaceutical formulation comprising:
a buffer;
an adeno-associated viral vector with a transgene encoding a microRNA; and
a cyclodextrin or a derivative thereof.

2. The isotonic formulation of claim 1, wherein the formulation has an osmolarity of from 250 to 330 mOsm/kg and a pH value of from 6.5 to 8.

3. The isotonic formulation of claim 1, wherein the formulation is essentially free of visible particles.

4. The isotonic formulation according to claim 1, wherein the adeno-associated viral vector with a transgene encoding a microRNA targets huntingtin mRNA.

5. The isotonic formulation according to claim 1, wherein the adeno-associated viral vector comprises an AAV2 serotype, an AAV5 serotype, an AAV9 serotype, a hybrid AAV serotype, or a combination thereof.

6. The isotonic formulation according to claim 1, wherein the concentration of the adeno-associated viral vector is up to 5E13 gc/ml.

7. The isotonic formulation according to claim 1, wherein the cyclodextrin is an unsubstituted or substituted β-cyclodextrin at a concentration of from about 0.05% w/v to about 2% w/v.

8. The isotonic formulation according to claim 1, further comprising a sugar or sugar alcohol at a concentration of from about 0.05% w/v to about 2% w/v.

9. The isotonic formulation of claim 8, wherein the sugar or sugar alcohol is selected from the group consisting of trehalose, sucrose, maltose, mannitol, and derivatives and combinations thereof.

10. The isotonic formulation according to claim 1, further comprising a pharmaceutically acceptable salt selected from the group consisting of NaCl, KCl, CaCl₂, MgCl₂, and combinations thereof, at a concentration of greater than 75 mM.

11. The isotonic formulation according to claim 1, wherein the buffer comprises a Tris buffer at a pH of about 7.5 to about 8.0.

12. The isotonic formulation according to claim 1, further comprising an amino acid selected from the group consisting of cysteine, arginine, histidine, glycine, and derivatives and combinations thereof, at a concentration of from about 2 mM to about 3 mM.

13. The isotonic formulation according to claim 1, wherein the formulation remains stable at room temperature (15° C.-25° C.) without substantial formation of aggregates or agglomerates for at least 12 hours after thawing.

14.-15. (canceled)

16. A method for treating Huntington's disease in a patient, the method comprising administering the isotonic formulation according to claim 1.

17. The method according to claim 16, wherein formulation is administered to the patient's brain by intrastriatal injection targeting the putamen and caudate nucleus.
